(19) United States
(12) Patent Application Publication
(10) Pub. No.: US 2023/0334386 A1
(43) Pub. Date: Oct. 19, 2023

(54) ACCESS CONTROL SYSTEM, ACCESS CONTROL METHOD, AND ACCESS CONTROL PROGRAM

(71) Applicant: HITACHI, LTD., Tokyo (JP)

(72) Inventors: Aika KAWAGOE, Tokyo (JP); Shinichi NAKAMURA, Tokyo (JP); Atsumi MANO, Tokyo (JP); Shingo MASUYAMA, Tokyo (JP)

(21) Appl. No.: 18/026,632

(22) PCT Filed: Sep. 22, 2021
(86) PCT No.: PCT/JP2021/034865

§ 371 Date: Mar. 16, 2023

(30) Foreign Application Priority Data
Jan. 28, 2021 (JP) 2021-012499

(51) Int. Cl. G06Q 10/0631; G06F 21/60

(52) U.S. Cl. CPC G06Q 10/06311; G06F 21/604

(57) ABSTRACT

An access control system includes: a model management unit that manages a chain of supply-related tasks as a model; a case data management unit that manages an execution history of the supply-related tasks as case data; an execution facility data management unit that manages identification information of a facility that performs the task as execution facility data in association with case identification information; a belonging facility management unit that manages a facility to which an executor of the task belongs; and an access control unit that refers to the execution facility data when access from the executor of the task is accepted, and permits access to a case associated with the facility to which the executor belongs. With such a configuration, detailed access control can be performed for the chain of the supply-related tasks.

### TECHNICAL FIELD

The present invention relates to an access control system, an access control method, and an access control program.

### BACKGROUND ART

In the related art, a technique described in PTL 1 is provided in order to control access from a user. This publication discloses that “a computer is caused to execute, when an application accesses a resource, a role specification procedure of specifying, from an operating environment in which the application is executed, a role stored in an operating environment storage unit that stores a role of a user in association with the operating environment in which the application is executed. In addition, a computer device disclosed in the present application causes the computer to execute an access control execution procedure of specifying, from an access control information storage unit that stores access control information in associated with the role of the user, access control information associated with the role specified by the role specification procedure, and executing access control to the resource based on the specified access control information”.

### CITATION LIST

### Patent Literature

PTL 1: JP2009-301357A

### SUMMARY OF INVENTION

### Technical Problem

In PTL 1, it is possible to deal with a situation where authorities granted to the same user are inconsistent due to access control by an operating system (OS) and access control by an application, but it is not possible to handle advanced access control in a system in which a plurality of users are involved in a complex manner.

For example, in a supply chain, which is a chain of supply-related tasks, a role is assigned to each user involved, and access control in a functional unit is performed for each role, but it is difficult to handle a case having a plurality of facilities that provide the same task. It is desired, for a case in which a task is executed at a certain facility, to restrict access from other facilities that provide the same task, or it is desired to permit access to a plurality of facilities that are in charge of tasks at different stages for the same case, and detailed and diverse access control is required.

Accordingly, an object of the invention is to provide an access control system, an access control method, and an access control program capable of performing detailed access control.

### Solution to Problem

In order to achieve the above object, one of a typical access control system, access control method, and access control program according to the invention manages a chain of supply-related tasks as a model, manages an execution history of the supply-related tasks as case data, manages identification information of a facility that performs the task as execution facility data in association with case identification information, manages a facility to which an executor of the task belongs, and refers to the execution facility data when access from the executor of the task is accepted, and permits access to a case associated with the facility to which the executor belongs.

### Advantageous Effects of Invention

According to the invention, detailed access control can be performed. Problems, configurations, and effects other than those described above will be clarified by the following description of embodiment.

### BRIEF DESCRIPTION OF DRAWINGS

FIG. 1 is a diagram illustrating access control according to an embodiment.

FIG. 2 is a configuration diagram of a computer that implements a system according to the embodiment.

FIG. 3 is a diagram (Part 1) illustrating specific examples of data.

FIG. 4 is a diagram (Part 2) illustrating specific examples of data.

FIG. 5 is a flowchart showing a processing procedure with reference to all cases.

FIG. 6 is a flowchart showing a processing procedure with reference to input data for a case.

FIG. 7 is a diagram illustrating role-and-tenant-based access control.

FIG. 8 is a specific example of tenant-and-task-based access control.

FIG. 9 is a diagram illustrating tenant management and a model structure.

### DESCRIPTION OF EMBODIMENTS

An embodiment will be described below with reference to the drawings.

### Embodiment

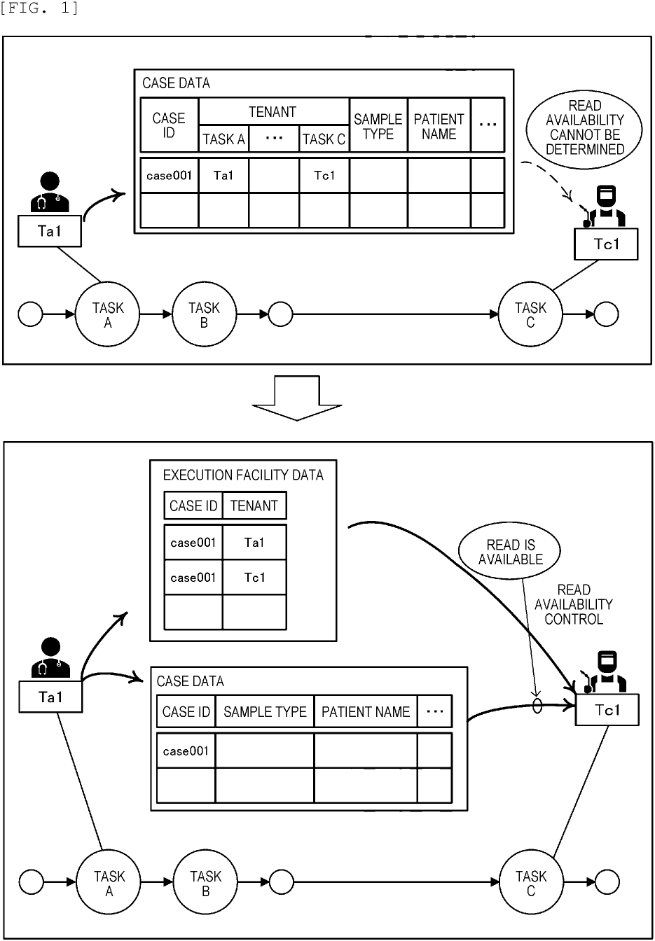

FIG. 1 is a diagram illustrating access control according to an embodiment. In this embodiment, access control in a system that manages a supply chain, which is a chain of supply-related tasks, in particular, a supply chain of regenerative medicine products and the like will be described as an example.

In the supply chain shown in FIG. 1, a task C is executed on an article obtained by sequentially executing a task A and a task B. Examples of the tasks include a task of collecting a sample from a patient, a task of transporting the sample, a task of producing a drug using the sample, a task of transporting the drug, and a task of administering the drug. In addition, the article is a sample and a drug at this time. In this system, the supply chain is managed by a model in which tasks and articles are set as nodes and the nodes are connected by links. By associating an execution history and the like with the nodes, a case-specific progress is managed.

Each task is not limited to be performed in a single facility, and one task may be performed in different facilities (tenants). For example, a patient may be sampled at a tenant Tal, which is a hospital, and another patient may be sampled at a tenant Ta2, which is another hospital.

In addition, the case-specific progress is managed by case data. The case data is data obtained by associating various information related to a case with a case ID for uniquely identifying the case.

Here, upon storing tenants in charge of the tasks in the case data, when a user other than the tenants in charge of the tasks accesses data of the tasks, availability of disclosure/non-disclosure of the data cannot be determined.

For example, when a user of a tenant Tc1 tries to access data of the task A of a case case001 performed by the tenant Ta1, “whether read is permitted” cannot be determined using tenant information.

Therefore, the system disclosed in this embodiment implements tenant-based access control for access to the case data by having, separately from the case data, execution facility data obtained by associating the case ID with the tenant.

In FIG. 1, in the execution facility data, the tenant “Ta1” and the tenant “Tc1” are associated with the case ID “case001”. Therefore, when the user of the tenant Tc1 tries to access to the data, by referring to the execution facility data and extracting the case ID of which the tenant Tc1 is in charge, the access to the case data for the case ID “case001” can be permitted.

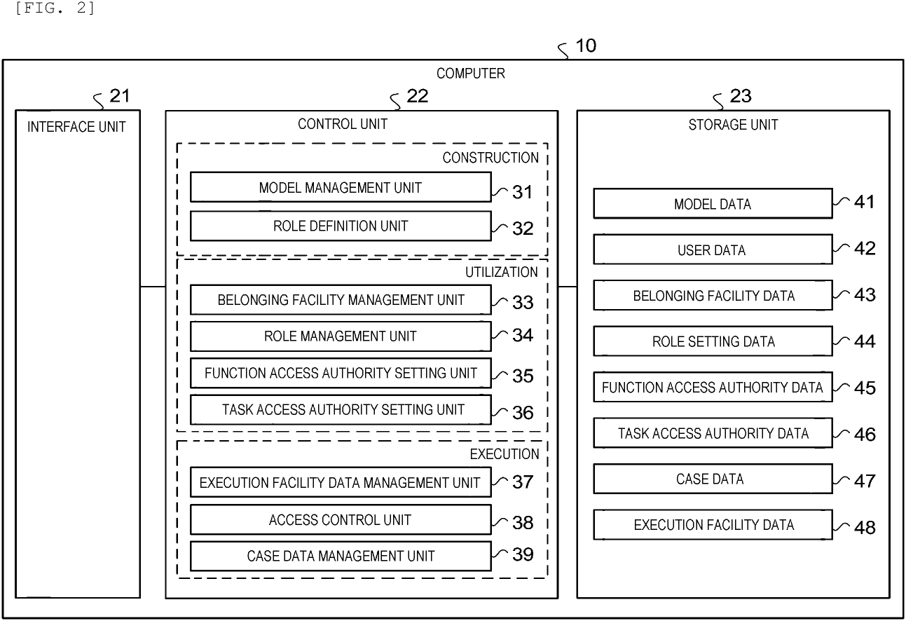

FIG. 2 is a configuration diagram of a computer that implements the system according to this embodiment. A computer 10 shown in FIG. 2 includes an interface unit 21, a control unit 22, and a storage unit 23.

The interface unit 21 receives input from an operator via, for example, a communication network, and provides an operation screen for the operator. The operator includes a constructor who constructs a supply chain model, a utilizer who sets access restrictions, and the like, and an executor who executes a task included in the supply chain.

The interface unit 21 can provide an individually provided operation screen for each of the constructor, the utilizer, and the executor.

The control unit 22 is, for example, a central processing unit (CPU), and implements various functions by loading programs in a memory (not shown) and executing the programs. The storage unit 23 is a storage medium such as a magnetic storage device, and is used to store various data.

Specifically, the control unit 22 operates as a model management unit 31, a role definition unit 32, a belonging facility management unit 33, a role management unit 34, a function access authority setting unit 35, a task access authority setting unit 36, an execution facility data management unit 37, an access control unit 38, and a case data management unit 39.

The storage unit 23 stores model data 41 representing the supply chain model, user data 42 obtained by registering a user who accesses the system, belonging facility data 43 obtained by assigning a belonging facility to the user, role setting data 44 obtained by assigning a role to the user, function access authority data 45 obtained by setting a function-specific access authority for the role, task access authority data 46 obtained by setting a task-specific access authority for the role, case data 47, which is information about a case-specific task or a case-specific article, and execution facility data 48 obtained by associating a facility with the case ID.

The model management unit 31 and the role definition unit 32 are provided for construction.

The model management unit 31 generates the supply chain model, and stores the generated model as the model data 41 in the storage unit 23 for management.

The role definition unit 32 defines a role that can be assigned to the user. The role is, for example, a doctor, a medical staff, a transportation personnel, or a pharmaceutical technician.

The belonging facility management unit 33, the role management unit 34, the function access authority setting unit 35, and the task access authority setting unit 36 are provided for utilization.

The belonging facility management unit 33 generates the belonging facility data 43 by associating the user including the executor of the task with a facility to which the user belongs, and registers the belonging facility data 43 in the storage unit 23, thereby managing the facility to which the user belongs.

The role management unit 34 generates the role setting data 44 by assigning the role to the user including the executor of the task, and registers the role setting data 44 in the storage unit 23.

The function access authority setting unit 35 sets the function-specific access authority for the role to generate the function access authority data 45, and stores the function access authority data 45 in the storage unit 23. Specifically, an API may be prepared for each function, and a role access authority may be set by each API.

The task access authority setting unit 36 sets the task-specific access authority for the role to generate the task access authority data 46, and stores the task access authority data 46 in the storage unit 23.

The execution facility data management unit 37, the access control unit 38, and the case data management unit 39 are provided for execution.

The execution facility data management unit 37 generates the execution facility data 48 by associating identification information of the facility that performs the task with case identification information, and stores the execution facility data 48 in the storage unit 23. Specifically, when a new case is to be registered, the execution facility data management unit 37 generates the execution facility data 48 by associating in advance an execution facility with each task of the new case.

When access is accepted from the user such as the executor of the task, the access control unit 38 performs access control based on a tenant and a role assigned to the user.

The case data management unit 39 manages and registers an execution history of each of the tasks of the supply chain in the case data 47.

Specifically, the access control unit 38 refers to the execution facility data 48 when the access from the executor of the task is accepted, and permits access to a case associated with the facility to which the executor belongs.

The access control unit 38 also accepts an operation permitted by both the function access authority data 45 and the task access authority data 46 for the case associated with the facility to which the executor belongs.

The computer 10 assigns a role to the executor of the task for management. For example, if the API is prepared for each function in the system, function-specific access control can be implemented for the role by setting the role access authority in each API. For the sake of convenience, such access control in unit of API is called API-based access control (ACL).

With only the API-based access control, the same access authority is given to any task. Therefore, the computer 10 further sets the task-specific access authority for the role, and performs function-and-task-based access control. For the sake of convenience, task-based access control is called process-based access control.

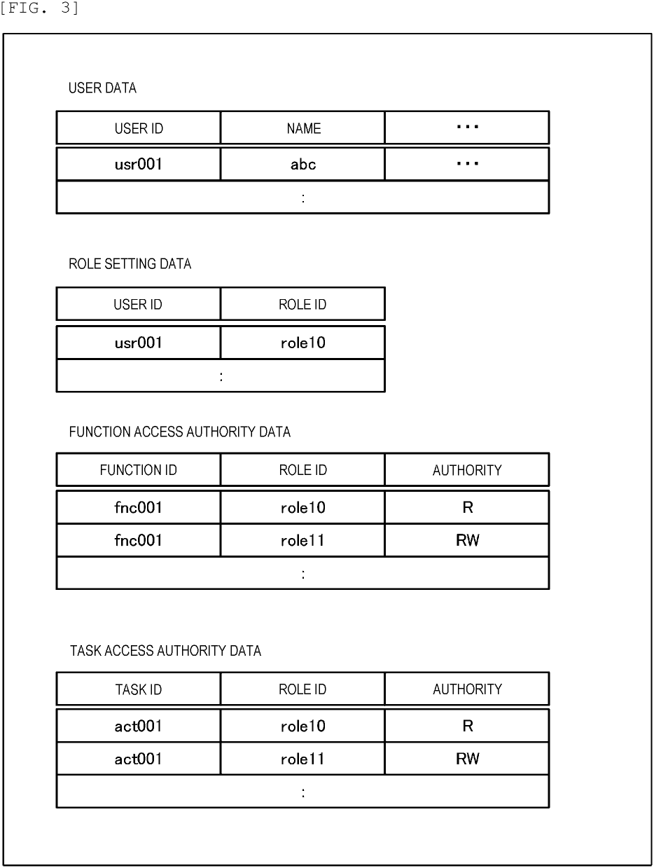

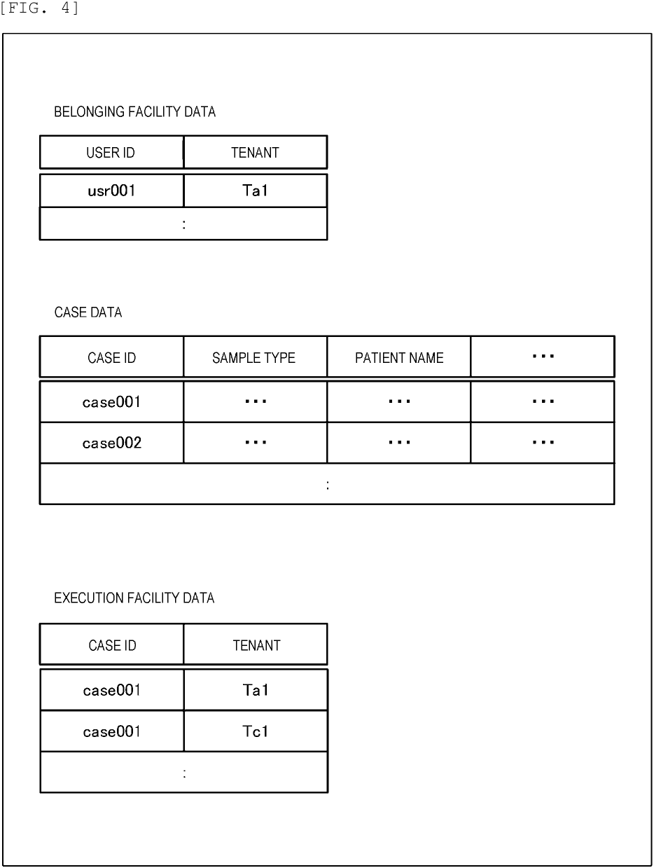

FIG. 3 and FIG. 4 are diagrams illustrating specific examples of data.

In the user data 42 shown in FIG. 3, information such as name is associated with user ID for identifying a user.

In the role setting data 44 shown in FIG. 3, role ID for specifying a role is associated with the user ID.

In the function access authority data 45 shown in FIG. 3, the role ID and authority are associated with function ID for specifying a function. The authority is designated by permission to read only (R), permission to read and write (RW), prohibition to both read and write (D), and the like.

In the task access authority data 46 shown in FIG. 3, the role ID and authority are associated with task ID for specifying a task. The authority is designated by permission to read only (R), permission to read and write (RW), prohibition to both read and write (D), and the like.

In the belonging facility data 43 shown in FIG. 4, tenant to which a user belongs is associated with the user ID for identifying a user.

In the case data 47 shown in FIG. 4, various information (sample type, patient name, and the like) related to a case are associated with case ID for uniquely identifying the case.

The execution facility data 48 shown in FIG. 4 is data obtained by associating facilities where tasks of a case are performed with the case ID. In FIG. 4, a combination of the case ID “case001” and the tenant “Ta1” and a combination of the case ID “case001” and the tenant “Tc1” are registered. In this way, when a plurality of tenants are involved in a case, combinations of the case ID and the tenant may be registered as many times as the number of the tenants involved.

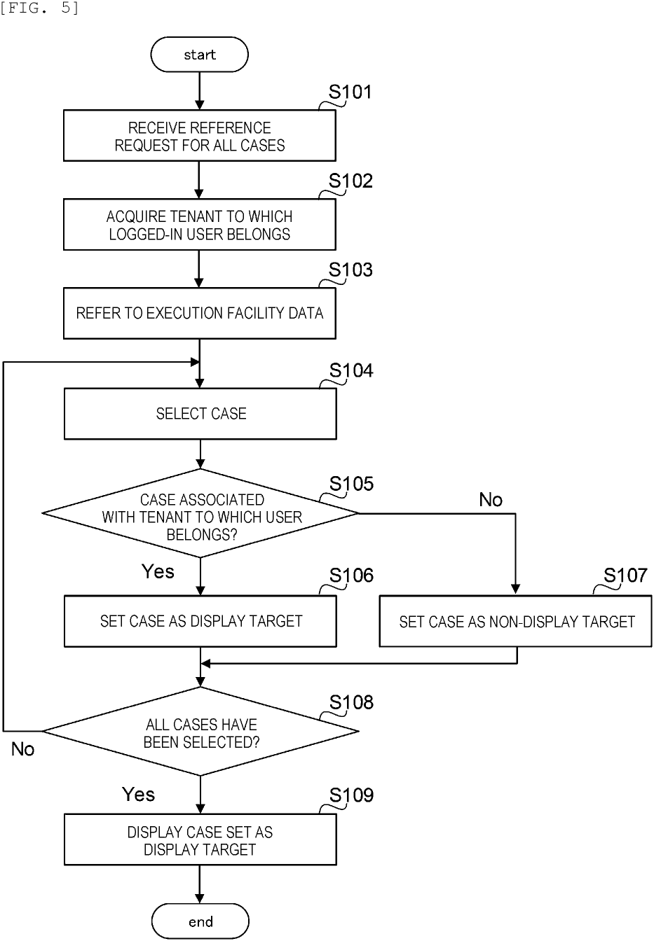

FIG. 5 is a flowchart showing a processing procedure with reference to all cases. The access control unit 38 receives a reference request for all cases (step S101), and then refers to the belonging facility data 43 to acquire a tenant to which a logged-in user belongs (step S102).

After step S102, the access control unit 38 refers to the execution facility data 48 (step S103) and selects a case (step S104). The access control unit 38 determines whether the selected case is a case associated with the tenant to which the user belongs (step S105).

As a result of the determination, if the case is a case associated with the tenant to which the user belongs (Yes at step S106), the access control unit 38 sets the case as a display target (step S106). On the other hand, if the case is not a case associated with the tenant to which the user belongs (No at step S106), the access control unit 38 sets the case as a non-display target (step S107).

After step S106 or step S107, the access control unit 38 determines whether all cases have been selected (step S108). If unselected cases remain (No at step S108), the access control unit 38 returns to step S104 to select another case.

If all cases have been selected (Yes at step S108), the access control unit 38 displays the case set as the display target (step S109), and ends the processing procedure in FIG. 5.

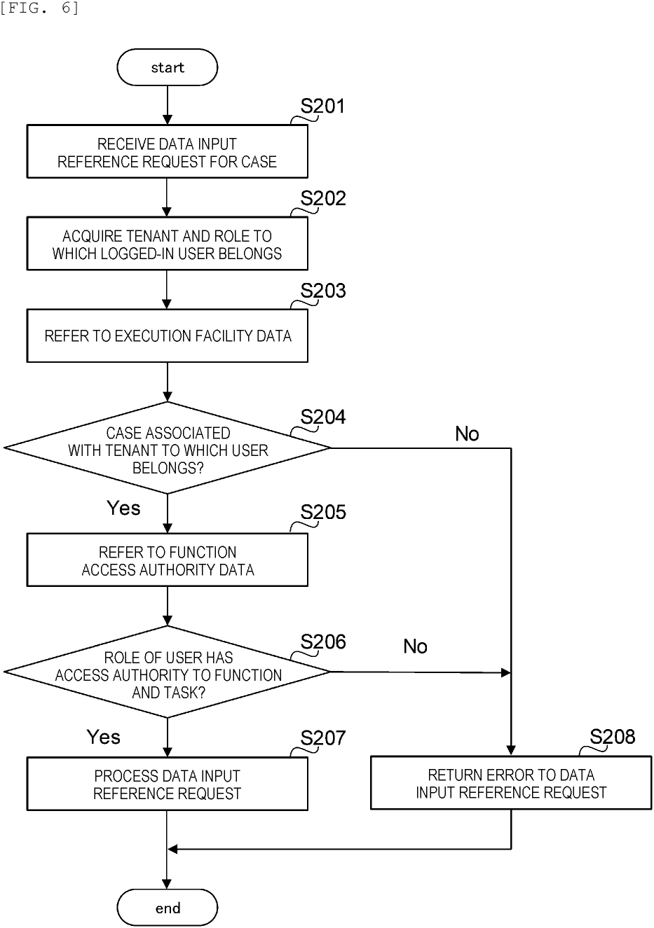

FIG. 6 is a flowchart showing a processing procedure with reference to input data for a case. The access control unit 38 receives a data input reference request for a case (step S201), and then refers to the belonging facility data 43 and the role setting data 44 to acquire a tenant and a role to which a logged-in user belongs (step S202).

After step S202, the access control unit 38 refers to the execution facility data 48 (step S203), and determines whether the case designated as a target of the data input reference request is a case associated with the tenant to which the user belongs (step S204). If the case is not a case associated with the tenant to which the user belongs (No at step S204), the access control unit 38 returns an error to the data input reference request (step S208), and ends the processing.

If the case is a case associated with the tenant to which the user belongs (Yes at step S204), the access control unit 38 refers to the function access authority data 45 and the task access authority data 46 (step S205), and determines whether a role of the user has an access authority to a function and a task (step S206).

If the role of the user has an access right to a function and a task (Yes at step S206), the access control unit 38 permits processing of the request, the case data management unit 39 processes the data input reference request (step S207), and the processing procedure in FIG. 6 is ended.

If the role of the user does not have an access right to a function or a task (No at step S206), the access control unit 38 returns an error to the data input reference request, and ends the processing procedure in FIG. 6.

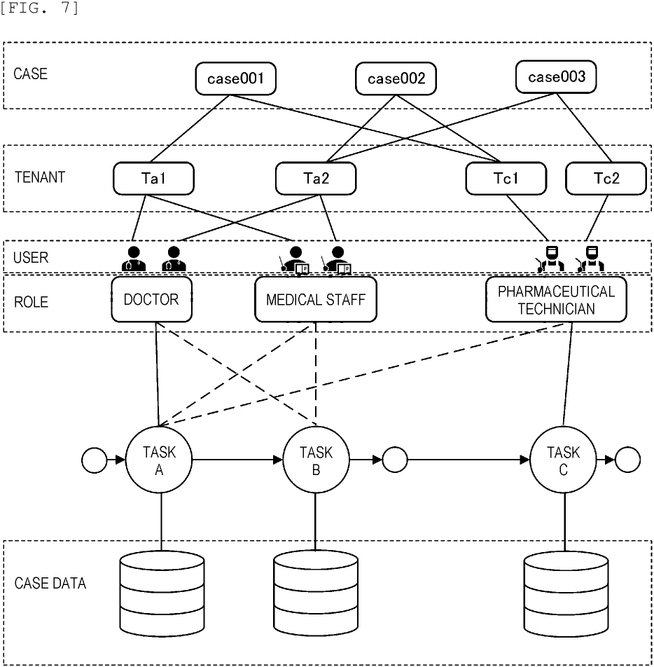

FIG. 7 is a diagram illustrating role-and-tenant-based access control. As shown in FIG. 7, a role and a tenant are associated with a user. A task-specific access authority in the supply chain is set for the role.

In FIG. 7, the role “doctor” is given a read and write authority for the task A and a read authority for the task B. In addition, the role “medical staff” is given a read authority for each of the task A and the task B. The role “pharmaceutical technician” is given a read authority for the task A and a read and write authority for the task C.

In addition, the case data is stored in association with the task in the supply chain model.

The case ID is associated with the tenant. In FIG. 7, the tenant Ta1 and the tenant Tc1 are associated with the case ID “case001”, and the tenant Ta2 and the tenant Tc1 are associated with a case ID “case002”. The tenant Ta2 and a tenant Tc2 are associated with a case ID “case003”.

In this way, by associating the case, the tenant, the user, the role, the model, and the case data with each other, the computer 10 implements tenant-and-task-based access control.

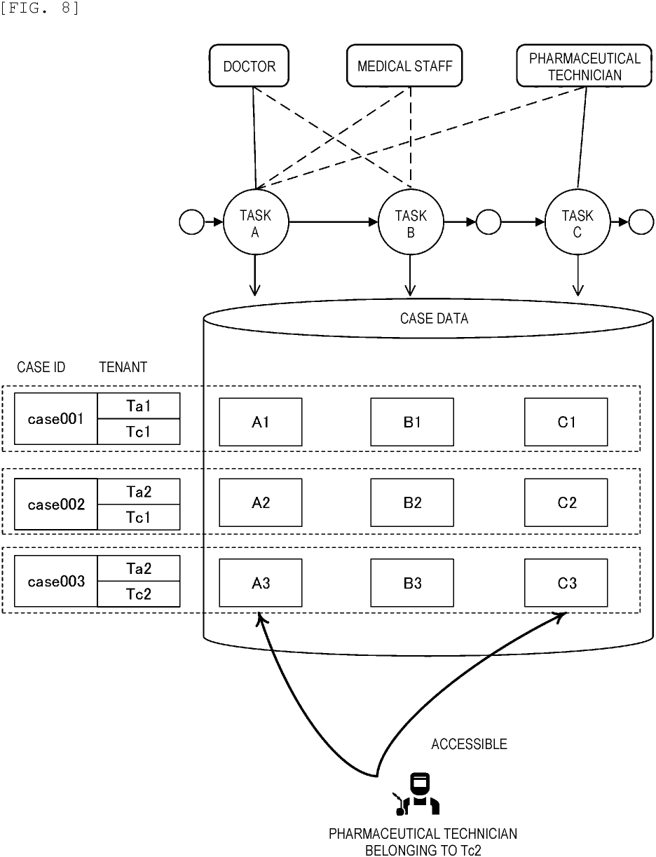

FIG. 8 is a specific example of the tenant-and-task-based access control. In FIG. 8, the tenants Ta1 and Tc1 are associated with the case ID “case001”, and data A1 as data of the task A, data B1 as data of the task B, and data C1 as data of the task C are also associated with the case ID “case001”.

In addition, the tenants Ta2 and Tc1 are associated with the case ID “case002”, and data A2 as the data of the task A, data B2 as the data of the task B, and data C2 as the data of the task C are also associated with the case ID “case002”.

In addition, the tenants Ta2 and Tc2 are associated with the case ID “case003”, and data A3 as the data of the task A, data B3 as the data of the task B, and data C3 as the data of the task C are also associated with the case ID “case003”.

Assuming that a pharmaceutical technician belonging to the tenant Tc tries to access these data, the data of the task A and the data of the task C permitted for the pharmaceutical technician, that is, the data A3 and the data C3 of the case ID “case003” associated with the tenant Tc2 can be accessed.

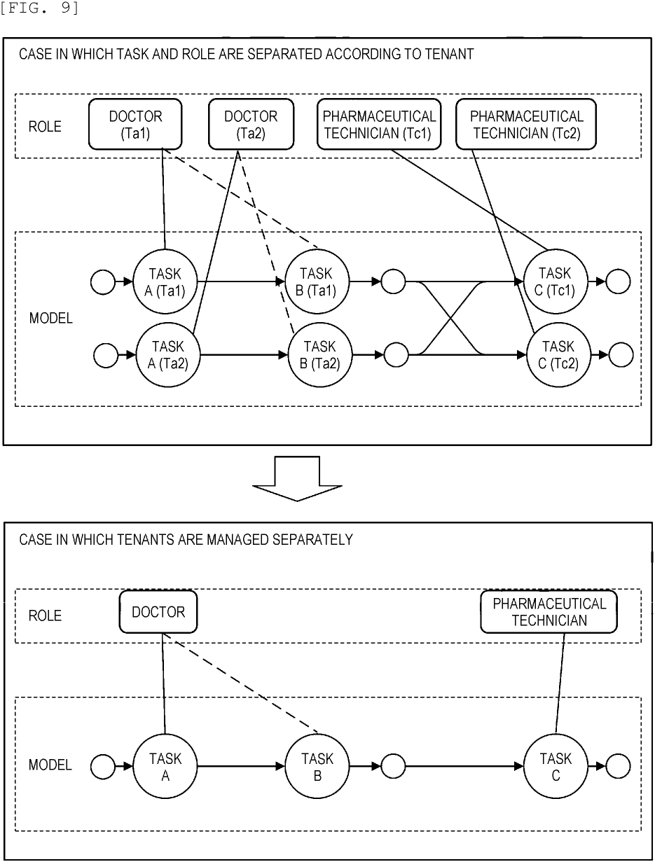

FIG. 9 is a diagram illustrating tenant management and a model structure. For example, by modeling the task A to be performed in the tenant Ta1 and the task A performed to be in the tenant Ta2 as separate tasks, and defining a doctor of the tenant Ta1 and a doctor of the tenant Ta2 as different roles, tenant-based access management can be performed, but in this case, the model structure becomes complicated due to the number of tenants, and addition or deletion of the tenants is not easy.

In contrast, managing the tenants as separate data simplifies the model structure and the role, and the addition and deletion of the tenants can also be handled.

As described above, the computer 10 that operates as an access control system according to this embodiment includes: the model management unit 31 that manages a chain of supply-related tasks as a model; the case data management unit 39 that manages an execution history of the supply-related tasks as case data; an execution facility data management unit 37 that manages identification information of a facility that performs the task as execution facility data in association with case identification information; the belonging facility management unit 33 that manages a facility to which an executor of the task belongs; and the access control unit 38 that refers to execution facility data when access from the executor of the task is accepted, and permits access to a case associated with the facility to which the executor belongs.

With such a configuration and operation, the access control system can restrict access from other facilities that provide the same task to a case in which a task is executed by a certain facility, and can permit access to a plurality of facilities that are in charge of tasks at different stages of the same case, and can implement detailed tenant-based access.

The computer 10 further includes the role management unit 34 that assigns a role to the executor of the task for management, and the function access authority setting unit 35 and the task access authority setting unit 36 as access authority setting units that set access an access authority for the role to generate access authority information. The access control unit 38 refers to the access authority information based on the role assigned to the executor, and accepts an operation permitted by the access authority information for a case associated with the facility to which the executor belongs.

With such a configuration and operation, it is possible to implement advanced access control obtained by combining the tenant, the task, and the function.

For example, it is possible to accept, for a case involved by any task, an operation permitted by both the task access authority information and the function access authority information.

In addition, when a new case is to be registered, the execution facility data management unit 37 associates in advance an execution facility with each task of the new case.

Therefore, it is possible to control access performed by an executor involved in each case.

In this embodiment, the management of the supply chain of the regenerative medicine products is exemplified, and the model management unit 31 manages a model for a chain of tasks including a task of collecting a sample from a patient, a task of transporting the sample, a task of producing a drug using the sample, a task of transporting the drug, and a task of administering the drug.

The supply chain of the regenerative medicine products is merely an example, and the invention can be used for any supply chain.

In this way, the configurations and operations shown in this embodiment are merely examples, and the invention can be implemented by appropriately changing the configurations and operations.

For example, the computer 10 does not necessarily have to be a device that physically has one housing, and operations similar to those of the computer 10 may be implemented by combining virtual resources.

### REFERENCE SIGNS LIST

10: computer; 21: interface unit; 22: control unit; 23: storage unit; 31: model management unit; 32: role definition unit; 33: belonging facility management unit; 34: role management unit; 35: function access authority setting unit; 36: task access authority setting unit; 37: execution facility data management unit; 38: access control unit; 39: case data management unit; 41: model data; 42: user data; 43: belonging facility data; 44: role setting data; 45: function access authority data; 46: task access authority data; 47: case data; 48: execution facility data

1. An access control system comprising:
a model management unit that manages a chain of tasks as a model;
a case data management unit that manages an execution history of the supply-related tasks as case data;
an execution facility data management unit that manages identification information of a facility that performs the task as execution facility data in association with case identification information;
a belonging facility management unit that manages a facility to which an executor of the task belongs; and
an access control unit that refers to the execution facility data when access from the executor of the task is accepted, and permits access to a case associated with the facility to which the executor belongs.

2. The access control system according to claim 1, further comprising:
a role management unit that assigns a role to the executor of the task for management; and
an access authority setting unit that sets an access authority for the role to generate access authority information, wherein
the access control unit refers to the access authority information based on the role assigned to the executor, and accepts an operation permitted by the access authority information for the case associated with the facility to which the executor belongs.

3. The access control system according to claim 2, wherein
the access authority setting unit generates function access authority information in which a function-specific access authority is set for the role and task access authority information in which task-specific access authority is set for the role, and
the access control unit accepts an operation permitted by both the task access authority information and the function access authority information.

4. The access control system according to claim 1, wherein
when a new case is to be registered, the execution facility data management unit associates in advance an execution facility with each task of the new case.

5. The access control system according to claim 1, wherein
the model management unit manages a model for a chain of tasks including a task of collecting a sample from a patient, a task of transporting the sample, a task of producing a drug using the sample, a task of transporting the drug, and a task of administering the drug.

6. An access control method comprising:
by a computer,
a model management step of managing a chain of supply-related tasks as a model;
a case data management step of managing an execution history of the supply-related tasks as case data;
an execution facility data management step of managing identification information of a facility that performs the task as execution facility data in association with case identification information;
a belonging facility management step of managing a facility to which an executor of the task belongs; and
an access control step of referring to the execution facility data when access from the executor of the task is accepted, and permitting access to a case associated with the facility to which the executor belongs.

7. An access control program, which causes a computer to execute:
a model management step of managing a chain of tasks as a model;
a case data management step of managing an execution history of the supply-related tasks as case data;
an execution facility data management step of managing identification information of a facility that performs the task as execution facility data in association with case identification information;
a belonging facility management step of managing a facility to which an executor of the task belongs; and
an access control step of referring to the execution facility data when access from the executor of the task is accepted, and permitting access to a case associated with the facility to which the executor belongs.
